(19) United States
(12) Patent Application Publication
(10) Pub. No.: US 2003/0202935 A1
(43) Pub. Date: Oct. 30, 2003

(54) Composition and formulations and their use as nociceptic, anti-axniolytic and anabolic agents

(76) Inventor: Jonathan W. Nyce, Princeton, NJ (US)

Correspondence Address: ALBERT P. Halluin, Howrey Simon Arnold & White, LLP, bOX 34, Menio park, CA, 94025, US

(21) Appl. No.: 09/554,485

(22) PCT Filed: Jan. 5, 2000
(86) PCT No.: PCT/US00/00180

(51) Int. Cl. A61K 51/00; A61K 49/00; A61K 31/57; A61K 31/525; A61K 31/192

(52) U.S. Cl. 424/1.11; 514/171; 514/251; 424/9.6; 514/161; 514/569; 514/570; 424/439

(57) ABSTRACT

Composition and formulations comprising a first agent such as folinic acid, pharmaceutically acceptable salts thereof or mixtures thereof, and a second agent(s) such as analgesics, muscle relaxants, mood disorder agents, anti-inflammatories, anti-migraine agents, anti-emetics, diuretics, high protein composites, and the like. The products are suitable as nociceptics and for the treatment of wasting disorders, bulimia, anorexia nervosa, anxiety, irritability and other symptoms associated with Pre Menstrual Syndrome, as well as for administration either in conjunction with steroids or to compensate adenosine depletion and/or bizarre behavior or aggression common in steroid users.

### TECHNICAL FIELD

This invention relates to a composition and formulations comprising folinic acid, pharmaceutically acceptable salts thereof or mixtures thereof, and other agents such as analgesics, anti-inflammatories, muscle relaxants, anti-migraine agents, anti-emetics, diuretics, and the like. The products are suitable as nociceptics, for treatment of wasting disorders, anxiety, irritability associated with Pre Menstrual Syndrome and for administration either in conjunction with steroids or to compensate adenosine depletion common in steroid users.

### BACKGROUND ART

Folinic acid is an intermediate product of the metabolism of folic acid, and is believed to be the active form into which folic acid is converted in the body. It is also known that ascorbic acid or Vitamin C is required as a necessary factor in the conversion of folic acid to folinic acid. Folinic acid has been used therapeutically as an antidote to folic acid antagonists such as methotrexate which block the conversion of folic acid into folinic acid. Additionally, folinic acid has been used as an anti-anemic, because of its ability to combat folate deficiency. Folinic acid has heretofore never been used in patients afflicted with adenosine depletion nor in a method to therapeutically elevate adenosine levels in the brain or other organ.

Adenosine is a purine involved in intermediary metabolism. Adenosine also participates in the regulation of physiological activity in a variety of mammalian tissues as well as in many local regulatory mechanisms, such as those occurring in synapses in the central nervous system (CNS) and at neuroeffector junctions in the peripheral nervous system. In the CNS, adenosine inhibits the release of a variety of neurotransmitters, such as acetylcholine, noradrenaline, dopamine, serotonin, glutamate and GABA, depresses neurotransmission, induces spinal analgesia possibly by reducing neuronal firing and possesses anxiolytic properties. In the heart, adenosine suppresses pacemaker activity, slows AV conduction, possesses antiarrhythmic and arrhythmogenic effects, modulates autonomic control and triggers the synthesis and release of prostaglandins. In addition, adenosine has potent vasodilatory effects and modulates vascular tone. At present, adenosine is used clinically for the treatment of SupraVentricular Tachycardia (SVT) and other cardiac anomalies, as well as for testing cardiovascular function. Adenosine analogues are also being investigated for use as anticonvulsant, anxiolytic and neuroprotective agents. Adenosine is also protective of tissues subjected to ischemia (oxygen deprivation) and aids reperfusion of these tissues, e. g. brain following stroke or other acute or chronic brain ischemia-producing conditions and diseases, heart following heart attack or other acute or chronic heart ischemia-producing conditions or diseases, and other organs at risk for ischemia associated with diseases and condition processes, acute and chronic physiological events and in transplantable organs during the harvest and transportation stages prior to tranplantation as well as in already transplanted organs, among others. Adenosine analogues also are being investigated for use as anti-convulsant, anxiolytic and neuroprotective agents. Adenosine, in addition, is a natural anti-inflammatory agent which, for example, is known to mediate the anti-inflammatory effect of methotrexate. Adenosine also promotes and accelerates wound healing and regulates neutrophil function via activation of a serine/threonine phosphatase. Although adenosine has various therapeutic applications as described above, it has an extremely short half life (about a second). Adenosine's short half life and its propensity to cause angina-like pain make it a poor choice for therapeutic applications.

In view of the foregoing, it is readily apparent that a large reduction in adenosine levels or adenosine depletion may lead to a broad variety of deleterious conditions, and that the ability to treat, reverse and prevent adenosine depletion is an extremely useful means of therapeutic intervention. An agent with a longer half life would provide a great advantage for use as a nociceptic and for the therapeutic and prophylactic treatment of a variety of diseases and conditions such as anxiety, irritability associated with Pre Menstrual Syndrome (PMS) and wasting disorders, and to counter the effects of steroids, among others.

### DISCLOSURE OF INVENTION

The present invention relates to a composition, formulations and a method of preventing and treating anxiety, nausea, irritability associated with Pre Menstrual Syndrome, wasting disorders, including bulimia and anorexia nervosa, as well as to increase muscle (protein) mass, which comprises administering to a subject in need of such treatment a pharmaceutical composition comprising a first agent selected from the group consisting of folinic acid, pharmaceutically acceptable salts thereof and mixtures thereof and a second agent such as other anti-anxiolytic agents, nociceptis, anti-depressants, anti-inflammatory agents and analgesics, among others. The composition may also comprise formulation ingredients and a physiologically acceptable carrier.

This method, thus, is applicable to the prophylactic and therapeutic treatment and reduction in severity of the diseases and conditions described above, regardless of their origin, or whether they are accompanied by a decrease in adenosine levels or not (whether due to endogenous abnormalities or the result of exogenously administered substances). Of particular importance is the application of the composition and formulations of the invention to the treatment and prevention of some diseases and conditions affecting the central nervous system (CNS) such as nausea, anxiety, symptoms of Pre Menstrual Syndrome, to treat CNS problems associated with steroids, wasting disease, to induce weight gain in anorexia nervosa and bulimia and to increase body mass generally, particularly muscle mass. The present agents may be provided as various pharmaceutical formulations in bulk or in unit form, as well as in the form of a foodstuff with other edible ingredients, e.g. energy bars, chewing gum, candy, drinks, cakes, salad dressings, pasta, etc.

### BEST MODE FOR CARRYING OUT THE INVENTION

The present invention arose from a desire by the inventor to improve on prior technology for the prevention and treatment of wasting disorders, anorexia nervosa or bulimia, anxiety, irritability associated with Pre Menstrual Syndrome, nausea, anxiety and other related pathologies which are often associated with adenosine depletion in the central nervous system (CNS). The present treatment is effective whether or not there is a marked adenosine reduction or depletion and whatever its cause, e.g. due to use of steroids and other adenosine depleting drugs, deficient adenosine synthesis, high adenosine metabolism or any other cause.

Adenosine is known to be a natural agent provided with analgesic, anti-inflammatory, anti-ischemic, soporific and anti-anxiety properties. The present inventor found, unexpectedly, that the first agent also exhibits sustainable activity as a nociceptic, anti-anxiety agent, to treat and prevent detrimental symptoms associated with the intake of steroids, such as bizarre behavior, craziness and aggression. He also found through his research that the first agent has substantial activity for treating and preventing irritability and other abnormal mood behaviors associated with Pre Menstrual Syndrome, wasting syndrome (the first agent acts as an anabolic) and bulimia and anorexia nervosa, among others. In the particular case of Pre Menstrual Syndrome, the inventor found that the symptoms are often associated with cyclic alterations in adenosine levels, perhaps due to fluctuations in levels of neuronal steroids.

Because it has an extremely short half life (about a second), and because it exhibits a propensity to cause angina-like pain, adenosine itself is a poor choice for the treatment of anxiety, Pre Menstrual Syndrome (PMS) symptoms, wasting disorders, anorexia nervosa and bulimia, and to counter the detrimental side effects of steroidal intake. The inventor posited that an agent such as folinic acid, which is capable of causing the synthesis of adenosine, has a significantly longer half life than adenosine and which does not produce angina-like pain, is better suited for administration to subjects afflicted with these conditions.

Adenosine cannot be reasonably administered as a therapeutic, however, because it has an extremely short half life (about a second). This fact, and its propensity to cause angina-like pain make adenosine itself a poor choice for the treatment of pain and inflammation. An agent such as folinic acid, which is capable of causing the synthesis of adenosine, is better suited for administration to subjects afflicted with these conditions. The inventor has shown that the administration of folinic acid induces the de novo synthesis of adenosine. He showed that the oral administration of folinic acid causes a dramatic increase in adenosine levels in the brain in an animal model. Since folinic acid has a much longer half life than adenosine itself, and is not associated with the induction of angina-like pain, the first agent represents an unexpected improvement over adenosine for increasing brain adenosine levels and is thereby suitable as a nociceptic, anti-anxiolytic and anti-bulimic agent, as well as for the treatment of anorexia nervosa, wasting diseases, to increase body mass and to counter steroidal CNS side effects. Folinic acid, its salts and their mixtures are efficacious in the prevention and treatment of these and other syndromes and conditions, where an increase in the level of adenosine is of therapeutic value.

The agent of the invention is provided as a pharmaceutical composition in combination with agents currently used to treat the diseases, conditions, symptoms and syndromes described above, and to address other co-symptoms encountered in these patients. The present technology is of extreme utility in subjects where existing treatments are partially effective at best or where, although the treatment may have been effective initially, its efficacy has eroded with time. The present method is effective in stimulating adenosine synthesis and, thereby, treat subjects who, for example, have used or been administered steroids therapeutically, for treating or controlling anxiety, nausea and irritability (such as is the case in Pre Menstrual Syndrome), to increase weight gain and treat wasting disorders, and to prevent extreme weight loss in anorexia nervosa and bulimia. The term “adenosine depletion” is intended to encompass both diseases and conditions such as nausea, anxiety, weight loss and the like, which are associated with adenosine levels which are significantly reduced or depleted in one or more tissues, as compared to previous adenosine levels in the same subject or to a standard average level for the species (cut-off point), and conditions where adenosine levels are essentially the same as previous adenosine levels in that subject but, because of some other condition or alteration in that patient, a therapeutic benefit is achieved in the patient by increasing the adenosine levels as compared to previous levels. The present method is carried out, preferably, on patients where adenosine levels are reduced, e.g., by more than about 5%, about 10%, about 15%, about 20%, about 30% and more, to fully depleted as compared to previous adenosine levels in the subject. Although the present invention is primarily concerned with the treatment of human subjects, it also is employed for the treatment of vertebrates in general, particularly mammals. Among the animals treated may be domesticated and wild animals, large and small, for veterinarian purposes, and including house pets (cats, dogs and the like), zoo animals, race horses, farm animals (cows, sheep and the like) and many others.

Folinic acid and its pharmaceutically acceptable salts, hereafter sometimes referred to as “active compounds,” are known and may be made in accordance with known procedures. See generally, The Merck Index, Monograph No. 4141 (11th Ed. 1989); U.S. Pat. No. 2,741,608.

The second agent of the present composition may be one or more of a variety of therapeutic and diagnostic agents which are suitable for administration to humans and/or animals. Some of the categories of agents suitable for incorporation into the present composition and formulations are analgesics, pre-menstrual medications, anti-menopausal agents such as hormones and the like, anti-aging agents, anti-anxiolytic agents, nociceptic agents, mood disorder agents, anti-depressants, anti-bipolar mood agents, anti-schizophrenic agents, anti-cancer agents, alkaloids, blood pressure controlling agents, hormones, anti-inflammatory agents, other agents suitable for the treatment and prophylaxis of diseases and conditions associated or accompanied with pain and inflammation, such as arthritis, bums, wounds, chronic bronchitis, chronic obstructive pulmonary disease (COPD), inflammatory bowel disease such as Chron's disease and ulcerative colitis, autoimmune disease, muscle relaxants, steroids, soporific agents, anti-ischemic agents, anti-arrhythmic agents, contraceptives, vitamins, minerals, tranquilizers, neurotransmitter regulating agents, wound and bum healing agents, anti-angiogenic agents, cytokines, growth factors, anti-metastatic agents, antacids, anti-histaminic agents, anti-bacterial agents, anti-viral agents, anti-gas agents, agents for reperfusion injury, counteracting appetite suppressants, sun screens, emollients, skin temperature lowering products, radioactive phosphorescent and fluorescent contrast diagnostic and imaging agents, libido altering agents, bile acids, laxatives, anti-diarrheic agents, skin renewal agents, hair growth agents, etc.

Among the hormones are female and male sex hormones such as premarin, progesterone, androsterones and their analogues, thyroxine and glucocorticoids, among the libido altering agents are Viagra and other NO-level modulating agents, among the analgesics are over-the-counter medications such as ibuprofen, oruda, aleve and acetaminofen and controlled substances such as morphine and codeine, among the anti-depressants are tricyclics, MAO inhibitors and epinephrine, γ-amino butyric acid (GABA), dopamine and serotonin level elevating agents, e.g. Prozac, Amytryptilin, Wellbutrin and Zoloft, among the skin renewal agents are Retin-A, hair growth agents such as Rogaine, among the anti-inflammatory agents are non-steroidal anti-inflammatory drugs (NSAIDs) and steroids, among the soporifics are melatonin and sleep inducing agents such as diazepam, cytoprotective, anti-ischemic and head injury agents such as enadoline, and many others. Examples of agents in the different groups are provided in the following list. Examples of analgesics are Acetominophen, Anilerdine, Aspirin, Buprenorphine, Butabital, Butorpphanol, Choline Salicylate, Codeine, Dezocine, Diclofenac, Diflunisal, Dihydrocodeine, Elcatoninin, Etodolac, Fenoprofen, Hydrocodone, Hydromorphone, Ibuprofen, Ketoprofen, Ketorolac, Levorphanol, Magnesium Salicylate, Meclofenamate, Mefenamic Acid, Meperidine, Methadone, Methotrimeprazine, Morphine, Nalbuphine, Naproxen, Opium, Oxycodone, Oxymorphone, Pentazocine, Phenobarbital, Propoxyphene, Salsalate, Sodium Salicylate, Tramadol and Narcotic analgesics in addition to those listed above. See, Mosby's Physician's GenRx. Examples of anti-anxiety agents include Alprazolam, Bromazepam, Buspirone, Chlordiazepoxide, Chlormezanone, Clorazepate, Diazepam, Halazepam, Hydroxyzine, Ketaszolam, Lorazepam, Meprobamate, Oxazepam and Prazepam, among others. Examples of anti-anxiety agents associated with mental depression are Chlordiazepoxide, Amitriptyline, Loxapine Maprotiline and Perphenazine, among others. Examples of anti-inflammatory agents are non-rheumatic Aspirin, Choline Salicylate, Diclofenac, Diflunisal, Etodolac, Fenoprofen, Floctafenine, Flurbiprofen, Ibuprofen, Indomethacin, Ketoprofen, Magnesium Salicylate, Meclofenamate, Mefenamic Acid, Nabumetone, Naproxen, Oxaprozin, Phenylbutazone, Piroxicam, Salsalate, Sodium Salicylate, Sulindac, Tenoxicam, Tiaprofenic Acid, Tolmetin. Examples of anti-inflammatories for ocular treatment are Diclofenac, Flurbiprofen, Indomethacin, Ketorolac, Rimexolone (generally for post-operative treatment). Examples of anti-inflammatories for non-infectious nasal applications are Beclomethaxone, Budesonide, Dexamethasone, Flunisolide, Triamcinolone, and the like. Examples of soporifics (anti-insomnia/sleep inducing agents) such as those utilized for treatment of insomnia, are Alprazolam, Bromazepam, Diazepam, Diphenhydramine, Doxylamine, Estazolam, Flurazepam, Halazepam, Ketazolam, Lorazepam, Nitrazepam, Prazepam Quazepam, Temazepam, Triazolam, Zolpidem and Sopiclone, among others. Examples of sedatives are Diphenhydramine, Hydroxyzine, Methotrimeprazine, Promethazine, Propofol, Melatonin, Trimeprazine, and the like. Examples of sedatives and agents used for treatment of petit mal and tremors, among other conditions, are Amitriptyline HCl, Chlordiazepoxide, Amobarbital, Secobarbital, Aprobarbital, Butabarbital, Ethchiorvynol, Glutethimide, L-Tryptophan, Mephobarbital, MethoHexital Na, Midazolam HCl, Oxazepam, Pentobarbital Na, Phenobarbital, Secobarbital Na, Thiamylal Na, and many others. Agents used in the treatment of head trauma (Brain Injury/Ischemia) include Enadoline HCl (e.g. for treatment of severe head injury, orphan status, Warner Lambert). Examples of cytoprotective agents and agents for the treatment of menopause and menopausal symptoms are Ergotamine, Belladonna Alkaloids and Phenobarbitals. Examples of agents for the treatment of menopausal vasomotor symptoms are Clonidine, Conjugated Estrogens and Medroxyprogesterone, Estradiol, Estradiol Cypionate, Estradiol Valerate, Estrogens, conjugated Estrogens, esterified Estrone, Estropipate and Ethinyl Estradiol. Examples of agents for treatment of symptoms of Pre Menstrual Syndrome (PMS) are Progesterone, Progestin, Gonadotrophic Releasing Hormone, oral contraceptives, Danazol, Luprolide Acetate and Vitamin B6. Examples of agents for the treatment of emotional/psychiatric treatments are Tricyclic Antidepressants including Amitriptyline HCl (Elavil), Amitriptyline HCl, Perphenazine (Triavil) and Doxepin HCl (Sinequan). Examples of tranquilizers, anti-depressants and anti-anxiety agents are Diazepam (Valium), Lorazepam (Ativan), Alprazolam (Xanax), SSRI's (selective Serotonin reuptake inhibitors), Fluoxetine HCl (Prozac), Sertaline HCl (Zoloft), Paroxetine HCl (Paxil), Fluvoxamine Maleate (Luvox), Venlafaxine HCl (Effexor), Serotonin, Serotonin Agonists (Fenfluramine), and other over the counter (OTC) medications. Examples of anti-migraine agents are Imitrex and the like.

Pharmaceutically acceptable salts should be both pharmacologically and pharmaceutically acceptable. Such pharmacologically and pharmaceutically acceptable salts can be prepared as alkaline metal or alkaline earth salts, such as sodium, potassium or calcium salts, or the carboxylic acid group of folinic acid. The calcium salt of folinic acid is a preferred pharmaceutically acceptable salt. Organic salts and esters are also suitable for use with this invention. The active compounds are preferably administered to the subject as a pharmaceutical composition. Pharmaceutical compositions for use in the present invention include systemic and topical formulations and, among these, preferred are formulations suitable for inhalation, oral, rectal, vaginal, nasal, ophthalmic, otical, intracavitary, intraorgan, topical (including buccal, sublingual, dermal and intraocular), parenteral (including subcutaneous, intradermal, intramuscular, intravenous and intraarticular) and transdermal administration, among others. The compositions may conveniently be presented in single or multiple unit dosage forms as well as in bulk, and may be prepared by any of the methods which are well known in the art of pharmacy. The composition of the invention may also be provided in the form of a kit, whether already formulated or with instructions for its formulation and administration regime. The kit may also contain other agents, such as those described above and, for example, when for parenteral administration, they may be provided with a carrier in a separate container, where the carrier may be sterile. The present composition may also be provided in a separate container which may be sterile for addition of a liquid carrier prior to administration. See, e.g. U.S. Pat. No. 4,956,355; UK Patent No. 2,240,472; EPO Patent Application Serial No. 429,187; PCT Patent Application Serial No. 91/04030, the relevant preparatory and compound portions of which are incorporated by reference above. See, also Mortensen, S. A., et al., Int. J. Tiss. Reac. XII(3): 155-162 (1990); Greenberg, S., et al., J. Clin. Pharm. 30: 596-608 (1990); Folkers, K., et al., Proc. Nat'l. Acad. Sci. 87: 8931-8934 (1990), the relevant preparatory and compounding portions of which are also incorporated herein by reference. Formulations suitable for oral and parenteral administration are preferred, and inhalable preparations are also preferred. All methods include the step of bringing the active compound into association with a carrier which constitutes one or more accessory ingredients. In general, the formulations are prepared by uniformly and intimately bringing the active compound into association with a liquid carrier, a finely divided solid carrier, or both, and then, if necessary, shaping the product into desired formulations.

Compositions suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such compositions may be prepared by any suitable method of pharmacy which includes the step of bringing into association the active compound and a suitable carrier. In general, the compositions of the invention are prepared by uniformly and intimately admixing the active compound with a liquid or finely divided solid carrier, or both, and then, if necessary, shaping the resulting mixture. For example, a tablet may be prepared by compressing or molding a power or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispensing agent(s). Molded tablets may be made by molding, in a suitable machine, the powdered compound moistened with an inert liquid binder. Compositions for oral administration may optionally include enteric coatings known in the art to prevent degradation of the compositions in the stomach and provide release of the drug in the small intestine. Compositions suitable for buccal (sub-lingual) administration include lozenges comprising the active compound in a flavored base, usually sucrose and acacia or tragacanth and pastilles comprising the compound in an inert base such as gelation and glycerin or sucrose and acacia.

Compositions suitable for parenteral administration comprise sterile aqueous and non-aqueous injection solutions of the active compound, which preparations are preferably isotonic with the blood of the intended recipient. These preparations may contain antioxidants, buffers, bacteriostats and solutes which render the compositions isotonic with the blood of the intended recipient. Aqueous and non-aqueous sterile suspensions may include suspending agents and thickening agents. The compositions may be presented in unit-dose or multi-dose containers, for example sealed ampules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described.

Compositions suitable for topical application to the skin preferably take the form of an ointment, cream, lotion, paste, gel, spray, aerosol, or oil. Carriers which may be used include vaseline, lanoline, polyethylene glycols, alcohols, transdermal enhancers, and combinations of two or more thereof. Compositions suitable for transdermal administration may be presented as discrete patches adapted to remain in intimate contact with the epidermis of the recipient for a prolonged period of time. Compositions suitable for transdermal administration may also be delivered by iontophoresis (see, e.g., Pharmaceutical Research 3, 318 (1986)) and typically take the form of an optionally buffered aqueous solution of the active compound.

The active compound of this invention (first agent) is provided within broad amounts of the composition. For example, folinic acid, its salts and their mixtures may be contained in the composition in amounts of about 0.001%, about 1%, about 2%, about 5% to about 99.999%, about 98%, about 90%, about 40%, about 20%, about 10%, about 5% of the composition, preferably about 1 to about 99%, more preferably about 2% to 40%, and still more preferably about 2% to about 10% of the composition. These amounts may be adjusted when and if additional agents with overlapping activities are included as discussed above.

The dosage of the active compound may vary depending on age, weight, and condition of the subject. Treatment may be initiated with a small dosage which is less than the optimal dose of the first agent of the invention, be it folinic acid or one of its salts. The dose may be increased until a desired and/or optimal effect under the circumstances is reached. In general, the dosage is about 1, about 5, about 10, about 20, about 50 mg/kg body weight and up to about 100, about 200, about 500 or about 1000 mg/kg body weight. Currently, preferred are dosages of about 5 to about 500 mg/kg body weight of the subject, more preferred are dosages of about 10 to about 200 mg/kg, and still more preferred are dosages of about 50 to about 100 mg/kg body weight of the subject. Higher or lower doses, however, are also contemplated and are, therefore, within the confines of this patent. In general, the active agent is preferably administered at a concentration that will afford effective results without causing any unduly harmful or deleterious side effects, and may be administered either as a single unit dose, or if desired in convenient subunits administered at suitable times throughout the day. The second therapeutic or diagnostic agent(s) is (are) administered in amounts which are known in the art to be effective for the intended application. In cases where the second agent has an overlapping activity with the principal agent, i.e. folinic acid and its salts, the dose of one of the other or of both agents may be adjusted to attain a desirable effect without exceeding a dose range which avoids untoward side effects. Thus, for example, when other analgesic and anti-inflammatory agents are added to the composition, they may be added in amounts known in the art for their intended application or in doses somewhat lower that when administered by themselves.

In general, the present composition is provided in a variety of systemic and topical formulations. The systemic or topical formulations of the invention are selected from the group consisting of oral, intrabuccal, intrapulmonary, rectal, intrauterine, intradermal, topical, dermal, parenteral, intratumor, intracranial, buccal, sublingual, nasal, intramuscular, subcutaneous, intravascular, intrathecal, inhalable, transdermal, intraarticular, intracavitary, implantable, transdermal, iontophoretic, intraocular, ophthalmic, vaginal, otical, intravenous, intramuscular, intraglandular, intraorgan, intralymphatic, implantable, slow release and enteric coating formulations. The actual preparation and compounding of these different formulations is known in the art and need not be detailed here. The active compounds may be administered once or several times a day. The active compounds disclosed herein may be administered to the lungs of a subject by any suitable means, but are preferably administered by generating an aerosol comprised of respirable particles, the respirable particles comprised of the active compound, which particles the subject inhales, i.e., by inhalation administration. The respirable particles may be liquid or solid. Particles comprised of active compound for practicing the present invention should include particles of respirable size, that is, particles of a size sufficiently small to pass through the mouth and larynx upon inhalation and into the bronchi and alveoli of the lungs. In general, particles ranging from about 0.5 to about 10 microns in size, more particularly, less than about 5 microns in size, are respirable. Particles of non-respirable size which are included in the aerosol tend to deposit in the throat and be swallowed, and the quantity of non-respirable particles in the aerosol is preferably minimized. For nasal administration, a particle size in the range of 10-500 :m is preferred to ensure retention in the nasal cavity.

Liquid pharmaceutical compositions of active compound for producing an aerosol may be prepared by combining the active compound with a stable vehicle, such as sterile pyrogen free water. Solid particulate compositions containing respirable dry particles of micronized active compound may be prepared by grinding dry active compound with a mortar and pestle, and then passing the micronized composition through a 400 mesh screen to break up or separate out large agglomerates. A solid particulate composition comprised of the active compound may optionally contain a dispersant which serves to facilitate the formation of an aerosol. A suitable dispersant is lactose, which may be blended with the active compound in any suitable ratio, e.g., a 1 to 1 ratio by weight.

Aerosols of liquid particles comprising the active compound may be produced by any suitable means, such as with a nebulizer. See, e.g. U.S. Pat. No. 4,501,729. Nebulizers are commercially available devices which transform solutions or suspensions of the active ingredient into a therapeutic aerosol mist either by means of acceleration of a compressed gas, typically air or oxygen, through a narrow venturi orifice or by mens of ultrasonic agitation. Suitable compositions for use in nebulizer consist of the active ingredient in liquid carrier, the active ingredient comprising up to 40% w/w composition, but preferably less than 20% w/w carrier being typically water or a dilute aqueous alcoholic solution, preferably made isotonic with body fluids by the addition of, for example sodium chloride. Optional additives include preservatives if the composition is not prepared sterile, for example, methyl hydroxybenzoate, antioxidants, flavoring agents, volatile oils, buffering agents and surfactants.

Aerosols of solid particles comprising the active compound may likewise be produced with any sold particulate medicament aerosol generator. Aerosol generators for administering solid particulate medicaments to a subject product particles which are respirable, as explained above, and generate a volume of aerosol containing a predetermined metered dose of a medicament at a rate suitable for human administration. Examples of such aerosol generators include metered dose inhalers and insufflators.

The second agent(s) may be administered concurrently with the active compound, and may be an agent for preventing and treating sleeplessness, mood disorders, anxiety, irritability, wasting disorders, bulimia, anorexia nervosa, cancer, viral and microbial infections, heart conditions, ischemia, menopause, pain, inflammation, wounds and burns, muscle tension, low bone calcification, inflammatory diseases such as autoimmune diseases, COPD, and inflammatory bowel disease, and many more, inflammatory conditions, wound healing, ischemia and reperfusion injury, to treat and prevent steroid intake secondary effects and to improve body weight and increase muscle mass, preferably in the same composition as described above. The phrase “concurrently administering,” as used herein, means that the folinic acid or its salt and the second agent(s) are administered either: (a) simultaneously in time, and preferably by formulating the two together in a common pharmaceutical carrier; or (b) at different times during the course of a common treatment schedule. In the latter case, the two compounds are administered at times effective to complement their half lives and, thereby offset a reduction in peak level of one with an increasing level of the other and, thereby, counter balance any decrease in activity of one with an increase in activity of the other as a result of their alternate administration schedule. Thus the active compound may or may not be administered for a time sufficient to bring endogenous adenosine levels back to prior levels in the subject. If the present composition or formulations are administered for a time sufficient to replenish endogenous adenosine levels (if lowered with respect to prior levels in the same subject), then the folinic acid, its salts or their mixtures and the second agent are administered in amounts effective to increase adenosine levels to a desired level. Thereafter, the doses of the two or more agents may be reduced so as to maintain adenosine levels, whether the second agent has overlapping activity with the active compound or, if of different activity, the dose of the second agent may be reduced along with that of the active compound in cases of reduced risk of relapse. If the active compound is administered for a time sufficient to replenish endogenous adenosine levels, and this is attained, the continuation of treatment will depend on whether the adenosine levels are maintain in the absence of treatment or not. Moreover, whether the dose of the second agent(s) is reduced or not will depend on whether or not it is necessary to continue its administration or the subject remains stable. If the practitioner perceives a need to offset a future relapse, be it as a decrease in adenosine levels or even its depletion and/or a need or benefit from a continued administration of the second agent (s), the treatment may be continued with close monitoring.

The additional agents, examples of which are listed above, may be administered per se or in the form of pharmaceutically acceptable salts. When used in medicine, the salts of these agents should be pharmacologically and pharmaceutically acceptable, but non-pharmaceutically acceptable salts may conveniently be used to prepare the free active compound or pharmaceutically acceptable salts thereof and are not excluded from the scope of this invention. Such pharmacologically and pharmaceutically acceptable salts include, but are not limited to, those prepared from the hydrochloric, hydrobromic, sulphuric, nitric, phosphoric, maleic, acetic, salicylic, p-toluenesulfonic, tartaric, citric, methanesulphonic, formic, malonic, succinic, naphthalene-2-sulphonic and benzenesulphonic acids, among others. Pharmaceutically acceptable salts also may be prepared as alkaline metal or alkaline earth salts, such as sodium, potassium or calcium salts of the carboxylic acid group. The present pharmaceutical formulations, whether veterinary or human use, may comprise, in addition to the active compound and one or more additional agents, one or more pharmaceutically acceptable carriers, and optionally any other therapeutic ingredients suitable for specific types of diseases and conditions. The carrier(s) must be pharmaceutically acceptable in the sense of being compatible with the other ingredients of the formulation and not unduly deleterious to the recipient thereof.

Formulations of the present invention suitable for oral administration may be presented as discrete units such as capsules, cachets, tablets or lozenges, each containing a predetermined amount of the potentiating agent as a powder or granules or a suspension in an aqueous liquor or nonaqueous liquid such as a syrup, an elixir, an emulsion or a drought.

A tablet may be made by compression or molding, optionally with one or more accessory ingredients. Compressed tablets may be prepared by compressing in a suitable machine, with the active compound being in a free-flowing form such as a powder or granules which is optionally mixed with a binder, disintegrant, lubricant, inert diluent, surface active agent or dispersing agent. Molded tablets comprised of a mixture of the powdered active compound with a suitable carrier may be made by molding in a suitable machine.

A syrup may be made by adding the active compound to a concentrated aqueous solution of a sugar, for example sucrose to which may also be added any accessory ingredient(s). Such accessory ingredient(s) may include flavorings, suitable preservatives, an agent to retard crystallization of the sugar, and an agent to increase the solubility of any other ingredient, such as a polyhydric alcohol, for example glycerol or sorbitol.

Formulations suitable for parenteral administration conveniently comprise a sterile aqueous preparation of the active compound, which is preferably isotonic with the blood of the recipient.

Nasal spray formulations comprise purified aqueous solutions of the active compound with preservative agents and isotonic agents. Such formulations are preferably adjusted to a pH and isotonic state compatible with the nasal mucous membranes.

Formulations for rectal or vaginal administration may be presented as a suppository with a suitable carrier such as cocoa butter, or hydrogenated fats or hydrogenated fatty carboxylic acids.

Ophthalmic formulations are prepared by a similar method to the nasal spray, except that the pH and isotonic factors are preferably adjusted to match that of the eye. Otical formulations are generally prepared in viscous carriers, such as oils and the like, as is known in the art, so that they may be easily administered into the ear without spilling.

Topical formulations comprise the active compound dissolved or suspended in one or more media such as mineral oil, petroleum, polyhydroxy alcohols or other bases used for topical pharmaceutical formulations. The addition of other accessory ingredients, vide infra, may be desirable.

In addition to the aforementioned ingredients, the formulations of this invention may further include one or more accessory ingredient(s) selected from diluents, buffers, flavoring agents, binders, disintegrants, surface active agents, thickeners, lubricants, preservatives (including antioxidants) and the like. Other ingredients may also be utilized as is known in the art.

Having now generally described this invention, the same will be better understood by reference to certain specific examples, which are included herein for purposes of illustration only and are not intended to be limiting of the invention or any embodiment thereof, unless so specified.

### EXAMPLES

In the following examples, and throughout this patent, “DHEA” means dehydroepiandrosterone, “F.A.” means folinic acid, “M” means methyltestosterone, “s” means seconds, “mg” means milligrams, “kg” means kilograms, “kw” means kilowatts, “Mhz” means megahertz, and “nmol” means nanomoles.

### Examples 1 and 2

### In Vivo Effects of Folinic Acid & DHEA on Adenosine Levels

Young adult male Fischer 344 rats (120 grams) were administered dehydroepiandrosterone (DHEA) (300 mg/kg) or methyltestosterone (40 mg/kg) in carboxymethyl cellulose by gavage once daily for fourteen days. Folinic acid (50 mg/kg) was administered intraperitoneally once daily for fourteen days. On the fifteenth day, the animals were sacrificed by microwave pulse (1.33 kw, 2450 MHZ, 6.5 s) to the cranium, which instantly denatures all brain protein and prevents further metabolism of adenosine. Hearts were removed from animals and flash frozen in liquid nitrogen within 10 seconds of death. Liver and lungs were removed en bloc and flash frozen within 30 seconds of death. Brain tissue was subsequently dissected. Tissue adenosine was extracted, derivatized to 1, N6-ethenoadenosine and analyzed by high performance liquid chromatography (HPLC) using spectrofluorometric detection according to the method of Clark and Dar (J. of Neuroscience Methods 25:243 (1988)). Results of these experiments are summarized in Table 2 below. Results are expressed as the mean±SEM, with X p<0.05 compared to control group and Ø p<0.05 compared to DHEA or methyltestosterone-treated groups.

TABLE 1
In vivo Effect of DHEA, δ-1-methyltestosterone & Folinic Acid on
Adenosine Levels in various Rat Tissues
Intracellular Adenosine (nmol/mg protein)

| | Heart | Lung | Brain |
|---|---|---|---|
| Control | 10.6 ± 0.6 | 3.1 ± 0. | 0.5 ± 0.04 |
| | (n = 12) | (n = 6) | (n = 12) |
| DHEA | 6.7 ± 0.5 | 2.3 ± 0.3 | 0.19 ± 0.01 |
| (300 mg/kg) | (n = 12) | (n = 6) | (n = 12) |
| Methyltestosterone | 8.3 ± 1.0 | N.D. | 0.42 ± 0.06 |
| (40 mg/kg) | (n = 6) | | (n = 6) |
| Methyltestost. (M) | 6.0 ± 0.4 | N.D. | 0.32 ± 0.03 |
| (120 mg/kg) | (n = 6) | | (n = 6) |
| Folinic Acid (F.A.) | 12.4 ± 2.1 | N.D. | 0.72 ± 0.09 |
| (50 mg/kg) | (n = 5) | | (n = 5) |
| DHEA + F.A. | 11.1 ± 0.6 | N.D. | 0.55 ± 0.09 |
| (300 mg/kg;50 mg/kg) | (n = 5) | | (n = 5) |
| M + F.A. | 9.1 ± 0.4 | N.D. | 0.60 ± 0.06 |
| (120 mg/kg;50 mg/kg) | (n = 6) | | (n = 6) |

N.D. = Not Determined

1. A pharmaceutical composition, comprising
a first agent selected from the group consisting of folinic acid, physiologically acceptable salts thereof and mixtures thereof in an anti-wasting, anti-bulimic, anti-anorexia nervosa, anti-anxiolytic, anti-irritability or anti-steroid associated bizarre behavior or aggression effective amount; and
a second agent selected from the group consisting of analgesics, anti-Pre Menstrual Syndrome agents, anti-menopausal agents, anti-aging agents, other anti-anxiolytic agents, mood disorder agents, anti-depressants, anti-bipolar mood disorder agents, anti-schizophrenic agents, anti-migraine agents, anti-cancer agents, alkaloids, blood pressure controlling agents, hormones, anti-inflammatory agents, muscle relaxants, other nociceptic agents, steroids, soporific agents, anti-ischemic agents, anti-arrhythmic agents, contraceptives, vitamins, minerals, tranquilizers, neurotransmitter regulating agents, wound healing agents, anti-angiogenic agents, cytokines, growth factors, anti-metastatic agents, antacids, anti-histaminic agents, anti-bacterial agents, anti-viral agents, anti-gas agents, appetite suppressants, anti-wasting disorder agents, anti-bulimic agents, anti-anorexia nervosa agents, brain injury agents, heart attack agents, adenosine, adenosine releasing agents and adenosine receptor stimulating agents, sun screens, emollients, skin temperature lowering agents, radioactive phosphorescent and fluorescent contrast diagnostic and imaging agents, libido altering agents, bile acids, laxatives, anti-diarrheic agents, skin renewal agents and hair growth agents.

2. The composition of claim 1, further comprising a carrier.

3. The composition of claim 2, wherein the carrier comprises a physiologically acceptable carrier.

4. The composition of claim 3, wherein the physiologically acceptable carrier comprises a pharmaceutically acceptable carrier.

5. The composition of claim 2, wherein the carrier is selected from the group consisting of solid and liquid carriers.

6. The composition of claim 1, further comprising an agent selected from the group consisting of anti-oxidants, flavoring agents, coloring agents, aromatic agents, volatile oils, buffering agents, dispersants, surfactants, propellants and preservatives.

7. The composition of claim 4, in the form of a systemic or topical formulation.

8. The composition of claim 7, which is selected from the group consisting of oral, intrabuccal, intrapulmonary, rectal, intrauterine, intradermal, topical, dermal, parenteral, intratumor, intracranial, buccal, sublingual, nasal, intramuscular, subcutaneous, intravascular, intrathecal, inhalable, transdermal, intraarticular, intracavitary, implantable, transdermal, iontophoretic, intraocular, ophthalmic, vaginal, otical, intravenous, intramuscular, intraglandular, intraorgan, intralymphatic, implantable, slow release and enteric coating formulations.

9. The formulation of claim 8, which is an oral formulation selected from the group consisting of capsules, cachets, lozenges, tablets, powder, granules, solutions, suspensions and emulsions.

10. The formulation of claim 9, wherein the solutions and suspensions are selected from the group consisting of aqueous and non-aqueous liquid solutions and suspensions, and the emulsions are selected from the group consisting of oil-in-water and water-in-oil emulsions.

11. The formulation of claim 9, which is a buccal or sub-lingual formulation selected from the group consisting of
lozenges further comprising a flavoring agent selected from the group consisting of sucrose, acacia and tragacanth; and
pastilles further comprising an inert base selected from the group consisting of gelatin, glycerin, sucrose and acacia.

12. The oral formulation of claim 9, further comprising an enteric coating.

13. The formulation of claim 8, which is a parenteral formulation selected from the group consisting of injectable solutions or suspensions, which may further comprise anti-oxidants, buffers, bacteriostatic agents and solutes which render the solution or suspension isotonic with the blood of any intended recipient.

14. The parenteral formulation of claim 13, wherein the solutions and suspensions are selected from the group consisting of sterile aqueous and non-aqueous injection solutions and suspensions, which may further comprise suspending agents and thickening agents.

15. The parenteral formulation of claim 13, which is in a single or multi-unit dose provided in a container selected from the group consisting of sealed ampules and vials.

16. The composition of claim 7, in single or multi-unit dose form.

17. The composition of claim 7, in bulk.

18. The composition of claim 7, which is freeze-dried or lyophilized.

19. The formulation of claim 8, which is a topical formulation selected from the group consisting of ointments, creams, lotions, pastes, gels, sprays, aerosols and oils.

20. The topical formulation of claim 19, further comprising a carrier selected from the group consisting of vaseline, lanoline, polyethylene glycols, alcohols and trans-dermal enhancers.

21. The formulation of claim 8, which is a transdermal formulation.

22. The transdermal formulation of claim 21, which is in the form of a solution or suspension of the first agent, which may further comprise a buffer and one or more second agent

23. The formulation of claim 21, provided along with a transdermal delivery device.

24. The formulation of claim 23, wherein the device is a patch.

25. The formulation of claim 8, which is an inhalable formulation.

26. The inhalable formulation of claim 25, which is an aerosol comprising liquid or solid particles of the agent, and which may further comprise an agent selected from the group consisting of preservatives, antioxidants, flavoring agents, coloring agents, aromatic agents, volatile oils, buffering agents, dispersants and surfactants.

27. The composition of claim 7, wherein the carrier comprises a hydrophobic carrier.

28. The formulation of claim 27, provided in a capsule.

29. The formulation of claim 8, which is a suppository.

30. The formulation of claim 8, which is an implant.

31. The formulation of claim 8, which is an slow release formulation.

32. The formulation of claim 8, which is an ophthalmic formulation.

33. The formulation of claim 8, which is an otical formulation.

34. The formulation of claim 8, which is a vaginal formulation selected from the group consisting of creams, gels, and vaginal suppositories and implants.

35. The composition of claim 2, comprising the first agent, a second agent selected from the group consisting of other analgesic agents, anti-inflammatory agents, muscle relaxant agents, anti-migraine agents, anti-depressants and other mood altering agents, vitamins, minerals, proteins and physiologically acceptable carriers.

36. The composition of claim 35, wherein the second agent comprises an analgesic.

37. The composition of claim 36, wherein the analgesic is selected from the group consisting of acetaminophen, salicylic acid, salts or esters thereof, naproxin, ibuprofen and narcotic analgesics.

38. The composition of claim 35, wherein the second agent comprises an anti-depressant and/or an anti-migraine agent.

39. The composition of claim 35, wherein the second agent comprises a barbiturate and/or an anti-migraine agent.

40. The composition of claim 35, wherein the second agent comprises a muscle relaxant.

41. The composition of claim 35, wherein the second agent comprises an anti-Pre Menstrual Syndrome agent.

42. The composition of claim 4, in the form of a foodstuff further comprising other edible ingredients.

43. The composition of claim 42, wherein the foodstuff is selected from the group consisting of energy bars, chewing gum, candy, drinks, cakes, pasta and salad dressings.

44. The composition of claim 7, which is an iontophoretic transdermal formulation, wherein the carrier is selected from the group consisting of aqueous and alcoholic solutions, oily solutions and suspensions and oil-in-water and water-in-oil emulsions, and wherein the formulation further comprises a transdermal transport promoting agent.

45. The formulation of claim 44, in the form of an implantable capsule or cartridge.

46. The composition of claim 4, wherein the carrier comprises a hydrophobic carrier.

47. The composition of claim 46, wherein the carrier comprises lipid vesicles or particles.

48. The composition of claim 47, wherein the vesicles comprise liposomes, and the particles comprise microcrystals.

49. The composition of claim 48, wherein the vesicles comprise liposomes which comprise the first agent, and further comprising optionally a second agent.

50. The composition of claim 49, wherein the vesicles comprise N-(1[2,3-dioleoxyloxi]propyl)-N,N,N-trimethyl-ammonium methylsulfate.

51. The composition of claim 8, comprising a respirable or inhalable formulation.

52. The formulation of claim 51, comprising an intrapulmonary formulation.

53. The formulation of claim 51, in the form of an aerosol.

54. The composition of claim 7, in the form of a kit, further comprising in a separate container, a delivery device; and instructions for addition of a carrier and administration.

55. The composition of claim 54, wherein the delivery device comprises an inhalator which delivers individual pre-metered doses of the formulation.

56. The composition of claim 54, wherein the inhalator comprises a nebulizer or insufflator, and further comprising a piercable or openable capsule or cartridge with solid particles of the composition.

57. The composition of claim 54, wherein the delivery device comprises a pressurized inhaler, and the formulation comprises a suspension or solution in an aqueous or non-aqueous liquid or an oil-in-water or water-in-oil emulsion.

58. A method of preventing or countering anxiety and/or irritability, comprising administering to a subject prone to or afflicted with anxiety and/or irritability, the pharmaceutical composition of claim 1, and optionally a carrier, the composition comprising an anti-anxiety and/or anti-irritability effective amount of the first agent.

59. The method of claim 58, wherein the first agent is administered in an amount of about 1 to about 1,000 mg/kg body weight.

60. The method of claim 59, wherein the agent is administered in an amount of about 5 to about 500 mg/kg body weight.

61. The method of claim 58, wherein the second agent selected from the group consisting of analgesics, anti-Pre Menstrual Syndrome agents, anti-menopausal agents, anti-aging agents, anti-anxiolytic agents, mood disorder agents, anti-depressants, anti-bipolar mood disorder agents, anti-schizophrenic agents, anti-migraine agents, other nociceptic agents, anti-cancer agents, alkaloids, blood pressure controlling agents, hormones, anti-inflammatory agents, muscle relaxants, steroids, soporific agents, anti-ischemic agents, anti-arrhythmic agents, contraceptives, vitamins, minerals, tranquilizers, neurotransmitter regulating agents, wound healing agents, anti-angiogenic agents, cytokines, growth factors, anti-metastatic agents, antacids, anti-histaminic agents, anti-bacterial agents, anti-viral agents, anti-gas agents, appetite suppressants, anti-wasting disorder agents, anti-bulimic agents, anti-anorexia nervosa agents, brain injury agents, heart attack agents, adenosine, adenosine releasing agents and adenosine receptor stimulating agents, sun screens, emollients, skin temperature lowering agents, radioactive phosphorescent and fluorescent contrast diagnostic and imaging agents, libido altering agents, bile acids, laxatives, anti-diarrheic agents, skin renewal agents and hair growth agents.

62. The method of claim 61, wherein the second agent comprises a mood disorder agent.

63. The method of claim 61, wherein the second agent comprises a muscle relaxant.

64. The method of claim 58, wherein the composition is administered by a systemic or topical route.

65. The method of claim 58, wherein the systemic or topical route is selected from the group consisting of oral, inhalable, topical, parenteral, and transdermal.

66. The method of claim 65, wherein the route of administration is selected from the group consisting of buccal, sublingual, dermal, intraocular, subcutaneous, intradermal, intramuscular, intravenous, intraarticular, intrapulmonary, rectal, intrauterine, intradermal, topical, dermal, parenteral, intratumor, intracranial, buccal, nasal, intravascular, intrathecal, inhalable, transdermal, intracavitary, implantable, transdermal, iontophoretic, intraocular, ophthalmic, vaginal, otical, intraglandular, intraorgan, intralymphatic and implantable.

67. The method of claim 66, wherein the composition is administered as an oral formulation selected from the group consisting of capsules, cachets, lozenges, tablets, powder, granules, solutions, suspensions and emulsions.

68. The method of claim 67, wherein the oral formulation further comprises an enteric coating.

69. The method of claim 65, wherein the composition is administered as a buccal or sub-lingual formulation selected from the group consisting of
lozenges further comprising a flavoring agent selected from the group consisting of sucrose, acacia and tragacanth; and
pastilles further comprising an inert base selected from the group consisting of gelatin, glycerin, sucrose and acacia.

70. The method of claim 65, wherein the composition is administered as a parenteral formulation selected from the group consisting of injectable solutions or suspensions, which may further comprise antioxidants, buffers, bacteriostatic agents and solutes which render the solution or suspension isotonic with the blood of any intended recipient.

71. The method of claim 70, wherein the parenteral formulation is provided in bulk or multi-dose form selected from the group consisting of sealed ampules and vials.

72. The method of claim 58, in unit-dose form.

73. The method of claim 58, wherein the formulation is in bulk or multi-dose form.

74. The method of claim 65, wherein the formulation is freeze-dried or lyophilized; and the method further comprises adding a sterile liquid carrier selected from the group consisting of saline and water prior to use.

75. The method of claim 65, wherein the composition is administered as a topical formulation selected from the group consisting of ointments, creams, lotions, pastes, gels, sprays, aerosols and oils; which may further comprise a carrier selected from the group consisting of vaseline, lanoline, polyethylene glycols, alcohols and trans-dermal enhancers.

76. The method of claim 65, wherein the composition is administered as a transdermal formulation in the form of a patch.

77. The method of claim 65, wherein the composition is administered as an iontophoretic formulation comprising a solution or suspension of the agent, and optionally a buffer and a second agent.

78. The method of claim 65, wherein the composition is administered as an inhalable formulation.

79. The method of claim 78, wherein the inhalable formulation is an aerosol comprising liquid or solid particles of the agent, and which may further comprise an agent selected from the group consisting of preservatives, antioxidants, flavoring agents, volatile oils, buffering agents, dispersants and surfactants.

80. The method of claim 58, wherein the composition further comprises an agent selected from the group consisting of physiologically acceptable carriers, preservatives, anti-oxidants, flavoring agents, volatile oils, buffering agents, dispersants and surfactants.

81. The method of claim 80, wherein the physiologically acceptable salt is selected from the group consisting of alkaline metal, alkaline earth salts and organic salts.

82. The method of claim 81, wherein the physiologically acceptable salts of the agents are selected from the group consisting of sodium, potassium, calcium and carboxylic acid salts.

83. The method of claim 58, wherein the subject is a human.

84. The method of claim 58, wherein the subject is an animal.

85. The method of claim 58, wherein, which is a prophylactic method.

86. The method of claim 58, which is a therapeutic method.

87. The method of claim 58, wherein the anxiety and/or irritability is (are) associated with Pre Menstrual Syndrome.

88. A method of increasing body mass, comprising administering to a subject in need of the treatment a pharmaceutical composition comprising a first agent selected from the group consisting of folinic acid, salts thereof and mixtures thereof, and optionally a physiologically acceptable carrier, the composition comprising a body mass increasing effective amount of the first agent.

89. The method of claim 88, wherein the patient is prone to or is afflicted with bulimia, anorexia nervosa and/or a wasting disorder.

90. The method of claim 88, wherein the patient has taken or is in need of taking steroids.

91. The method of claim 88, wherein the first agent is administered in an amount of about 1 to about 1,000 mg/kg body weight.

92. The method of claim 91, wherein the agent is administered in an amount of about 5 to about 500 mg/kg body weight.

93. The method of claim 88, wherein the second agent selected from the group consisting of analgesics, anti-Pre Menstrual Syndrome agents, anti-menopausal agents, anti-aging agents, anti-anxiolytic agents, mood disorder agents, nociceptic agents, anti-migraine agents, anti-depressants, anti-bipolar mood disorder agents, anti-schizophrenic agents, anti-cancer agents, alkaloids, blood pressure controlling agents, hormones, anti-inflammatory agents, muscle relaxants, steroids, soporific agents, anti-ischemic agents, anti-arrhythmic agents, contraceptives, vitamins, minerals, tranquilizers, neurotransmitter regulating agents, wound healing agents, anti-angiogenic agents, cytokines, growth factors, anti-metastatic agents, antacids, anti-histaminic agents, anti-bacterial agents, anti-viral agents, anti-gas agents, appetite suppressants, anti-wasting disorder agents, anti-bulimic agents, anti-anorexia nervosa agents, brain injury agents, heart attack agents, adenosine, adenosine releasing agents and adenosine receptor stimulating agents, sun screens, emollients, skin temperature lowering agents, radioactive phosphorescent and fluorescent contrast diagnostic and imaging agents, libido altering agents, bile acids, laxatives, anti-diarrheic agents, skin renewal agents and hair growth agents.

94. The method of claim 93, wherein the second agent comprises a mood disorder agent.

95. The method of claim 93, wherein the second agent comprises a steroid.

96. The method of claim 88, wherein the composition is administered by a systemic or topical route.

97. The method of claim 88, wherein the systemic or topical route is selected from the group consisting of oral, inhalable, topical, parenteral, and transdermal.

98. The method of claim 97, wherein the route of administration is selected from the group consisting of buccal, sublingual, dermal, intraocular, subcutaneous, intradermal, intramuscular, intravenous, intraarticular, intrapulmonary, rectal, intrauterine, intradermal, topical, dermal, parenteral, intratumor, intracranial, buccal, nasal, intravascular, intrathecal, inhalable, transdermal, intracavitary, implantable, transdermal, iontophoretic, intraocular, ophthalmic, vaginal, otical, intraglandular, intraorgan, intralymphatic and implantable.

99. The method of claim 98, wherein the composition is administered as an oral formulation selected from the group consisting of capsules, cachets, lozenges, tablets, powder, granules, solutions, suspensions and emulsions.

100. The method of claim 99, wherein the oral formulation further comprises an enteric coating.

101. The method of claim 97, wherein the composition is administered as a buccal or sub-lingual formulation selected from the group consisting of
lozenges further comprising a flavoring agent selected from the group consisting of sucrose, acacia and tragacanth; and
pastilles further comprising an inert base selected from the group consisting of gelatin, glycerin, sucrose and acacia.

102. The method of claim 97, wherein the composition is administered as a parenteral formulation selected from the group consisting of injectable solutions or suspensions, which may further comprise antioxidants, buffers, bacteriostatic agents and solutes which render the solution or suspension isotonic with the blood of any intended recipient.

103. The method of claim 102, wherein the parenteral formulation is provided in bulk or multi-dose form selected from the group consisting of sealed ampules and vials.

104. The method of claim 88, in unit-dose form.

105. The method of claim 88, wherein the formulation is in bulk or multi-dose form.

106. The method of claim 97, wherein the formulation is freeze-dried or lyophilized and the method further comprises adding a sterile liquid carrier selected from the group consisting of saline and water prior to use.

107. The method of claim 97, wherein the composition is administered as a topical formulation selected from the group consisting of ointments, creams, lotions, pastes, gels, sprays, aerosols and oils; which may further comprise a carrier selected from the group consisting of vaseline, lanoline, polyethylene glycols, alcohols and trans-dermal enhancers.

108. The method of claim 97, wherein the composition is administered as a transdermal formulation in the form of a patch.

109. The method of claim 97, wherein the composition is administered as an iontophoretic formulation comprising a solution or suspension of the agent, and optionally a buffer and a second agent.

110. The method of claim 97, wherein the composition is administered as an inhalable formulation.

111. The method of claim 110, wherein the inhalable formulation is an aerosol comprising liquid or solid particles of the agent, and which may further comprise an agent selected from the group consisting of preservatives, antioxidants, flavoring agents, volatile oils, buffering agents, dispersants and surfactants.

112. The method of claim 88, wherein the composition further comprises an agent selected from the group consisting of physiologically acceptable carriers, preservatives, anti-oxidants, flavoring agents, volatile oils, buffering agents, dispersants and surfactants.

113. The method of claim 112, wherein the physiologically acceptable salt is selected from the group consisting of alkaline metal, alkaline earth salts and organic salts.

114. The method of claim 113, wherein the physiologically acceptable salts of the agents are selected from the group consisting of sodium, potassium, calcium and carboxylic acid salts.

115. The method of claim 88, wherein the subject is a human.

116. The method of claim 88, wherein the subject is an animal.

117. The method of claim 88, which is a prophylactic method.

118. The method of claim 88, which is a therapeutic method.

119. A method of countering or preventing nausea, comprising administering to a subject in need of the treatment a pharmaceutical composition comprising a first agent selected from the group consisting of folinic acid, salts thereof and mixtures thereof, and optionally a physiologically acceptable carrier, the composition comprising a nociceptic effective amount of the first agent.

120. The method of claim 119, wherein the first agent is administered in an amount of about 1 to about 1,000 mg/kg body weight.

121. The method of claim 120, wherein the agent is administered in an amount of about 5 to about 500 mg/kg body weight.

122. The method of claim 119, wherein the second agent selected from the group consisting of analgesics, anti-Pre Menstrual Syndrome agents, anti-menopausal agents, anti-aging agents, anti-anxiolytic agents, mood disorder agents, nociceptic agents, anti-migraine agents, anti-depressants, anti-bipolar mood disorder agents, anti-schizophrenic agents, anti-cancer agents, alkaloids, blood pressure controlling agents, hormones, anti-inflammatory agents, muscle relaxants, steroids, soporific agents, anti-ischemic agents, anti-arrhythmic agents, contraceptives, vitamins, minerals, tranquilizers, neurotransmitter regulating agents, wound healing agents, anti-angiogenic agents, cytokines, growth factors, anti-metastatic agents, antacids, anti-histaminic agents, anti-bacterial agents, anti-viral agents, anti-gas agents, appetite suppressants, anti-wasting disorder agents, anti-bulimic agents, anti-anorexia nervosa agents, brain injury agents, heart attack agents, adenosine, adenosine releasing agents and adenosine receptor stimulating agents, sun screens, emollients, skin temperature lowering agents, radioactive phosphorescent and fluorescent contrast diagnostic and imaging agents, libido altering agents, bile acids, laxatives, anti-diarrheic agents, skin renewal agents and hair growth agents.

123. The method of claim 122, wherein the second agent comprises a mood disorder agent.

124. The method of claim 122, wherein the second agent comprises an anti-migraine agent, an anti-depressant, another nociceptic agent and/or a muscle relaxant.

125. The method of claim 119, wherein the composition is administered by a systemic or topical route.

126. The method of claim 119, wherein the systemic or topical route is selected from the group consisting of oral, inhalable, topical, parenteral, and transdermal.

127. The method of claim 126, wherein the route of administration is selected from the group consisting of buccal, sublingual, dermal, intraocular, subcutaneous, intradermal, intramuscular, intravenous, intraarticular, intrapulmonary, rectal, intrauterine, intradermal, topical, dermal, parenteral, intratumor, intracranial, buccal, nasal, intravascular, intrathecal, inhalable, transdermal, intracavitary, implantable, transdermal, iontophoretic, intraocular, ophthalmic, vaginal, otical, intraglandular, intraorgan, intralymphatic and implantable.

128. The method of claim 127, wherein the composition is administered as an oral formulation selected from the group consisting of capsules, cachets, lozenges, tablets, powder, granules, solutions, suspensions and emulsions.

129. The method of claim 128, wherein the oral formulation further comprises an enteric coating.

130. The method of claim 126, wherein the composition is administered as a buccal or sub-lingual formulation selected from the group consisting of
lozenges further comprising a flavoring agent selected from the group consisting of sucrose, acacia and tragacanth; and
pastilles further comprising an inert base selected from the group consisting of gelatin, glycerin, sucrose and acacia.

131. The method of claim 126, wherein the composition is administered as a parenteral formulation selected from the group consisting of injectable solutions or suspensions, which may further comprise antioxidants, buffers, bacteriostatic agents and solutes which render the solution or suspension isotonic with the blood of any intended recipient.

132. The method of claim 131, wherein the parenteral formulation is provided in bulk or multi-dose form selected from the group consisting of sealed ampules and vials.

133. The method of claim 119, in unit-dose form.

134. The method of claim 119, wherein the formulation is in bulk or multi-dose form.

135. The method of claim 126, wherein the formulation is freeze-dried or lyophilized and the method further comprises adding a sterile liquid carrier selected from the group consisting of saline and water prior to use.

136. The method of claim 126, wherein the composition is administered as a topical formulation selected from the group consisting of ointments, creams, lotions, pastes, gels, sprays, aerosols and oils; which may further comprise a carrier selected from the group consisting of vaseline, lanoline, polyethylene glycols, alcohols and trans-dermal enhancers.

137. The method of claim 126, wherein the composition is administered as a transdermal formulation in the form of a patch.

138. The method of claim 126, wherein the composition is administered as an iontophoretic formulation comprising a solution or suspension of the agent, and optionally a buffer and a second agent.

139. The method of claim 126, wherein the composition is administered as an inhalable formulation.

140. The method of claim 139, wherein the inhalable formulation is an aerosol comprising liquid or solid particles of the agent, and which may further comprise an agent selected from the group consisting of preservatives, antioxidants, flavoring agents, volatile oils, buffering agents, dispersants and surfactants.

141. The method of claim 119, wherein the composition further comprises an agent selected from the group consisting of physiologically acceptable carriers, preservatives, anti-oxidants, flavoring agents, volatile oils, buffering agents, dispersants and surfactants.

142. The method of claim 141, wherein the physiologically acceptable salt is selected from the group consisting of alkaline metal, alkaline earth salts and organic salts.

143. The method of claim 142, wherein the physiologically acceptable salts of the agents are selected from the group consisting of sodium, potassium, calcium and carboxylic acid salts.

144. The method of claim 119, wherein the subject is a human.

145. The method of claim 119, wherein the subject is an animal.

146. The method of claim 119, which is a prophylactic method.

147. The method of claim 119, which is a therapeutic method.
